(19) United States
(12) Patent Application Publication
(10) Pub. No.: US 2015/0057496 A1
(43) Pub. Date: Feb. 26, 2015

(54) Wirelessly Powered Capsule Endoscope

(71) Applicant: WITRICITY CORPORATION, Watertown, MA (US)

(72) Inventors: David A. Schatz, Needham, MA (US); Steven J. Ganem, Medfield, MA (US); Colin McCarthy, Norwell, MA (US)

(21) Appl. No.: 14/469,256

(22) Filed: Aug. 26, 2014

Related U.S. Application Data
(60) Provisional application No. 61/869,795, filed on Aug. 26, 2013.

(51) Int. Cl. A61B 1/04; A61B 1/00

(52) U.S. Cl. CPC A61B 1/041; A61B 1/00108
USPC 600/102; 600/109

(57) ABSTRACT

The disclosure features wireless power transfer systems that include a wirelessly powered capsule endoscope comprising a camera, a light source, electronics, an antenna, a device resonator, and a capsule enclosure; and a power supply resonator; wherein the power supply resonator is configured and arranged to resonantly couple with the device resonator to provide power to the wirelessly powered capsule endoscope via an oscillating magnetic field.

### CROSS-REFERENCE TO RELATED APPLICATIONS

This application claims priority to U.S. Provisional Patent Application No. 61/869,795, filed on Aug. 26, 2013, the entire contents of which are incorporated herein by reference.

### TECHNICAL FIELD

This disclosure relates to wireless power transfer.

### BACKGROUND

Energy or power may be transferred wirelessly using a variety of known radiative, or far-field, and non-radiative, or near-field, techniques as detailed, for example, in commonly owned U.S. patent application Ser. No. 12/613,686 published on May 6, 2010 as US 2010/0109445 and entitled “Wireless Energy Transfer Systems,” U.S. patent application Ser. No. 12/860,375 published on Dec. 9, 2010 as 2010/0308939 and entitled “Integrated Resonator-Shield Structures,” U.S. patent application Ser. No. 13/222,915 published on Mar. 15, 2012 as 2012/0062345 and entitled “Low Resistance Electrical Conductor,” the contents of which are incorporated by reference.

Wireless energy transfer may be difficult to incorporate or deploy in many environments. Efficiency of energy transfer, practicality, safety, and cost are factors that can prohibit the deployment for many applications. Therefore a need exists for a wireless energy transfer that addresses such practical challenges to allow widespread use of wireless energy transfer in various user environments.

### SUMMARY

Highly resonant wireless power transfer systems may include high quality factor resonators that may be driven to generate oscillating electromagnetic fields and that may interact with oscillating magnetic fields to generate currents and/or voltages in electronic circuits. That is, energy may be transferred wirelessly using oscillating magnetic fields. Resonators and electronics may be integrated or located inside of endoscopes, capsule endoscopes, medical imaging tools, and the like. A capsule endoscope may be powered wirelessly. A wirelessly powered capsule endoscope may be self-contained with no wired connections between the capsule endoscope and the source of power.

In general, in a first aspect, the disclosure features wireless power transfer systems that include a wirelessly powered capsule endoscope comprising a camera, a light source, electronics, an antenna, a device resonator, and a capsule enclosure; and a power supply resonator; wherein the power supply resonator is configured and arranged to resonantly couple with the device resonator to provide power to the wirelessly powered capsule endoscope via an oscillating magnetic field.

The power supply resonator can comprise a source resonator and repeater resonators, and the system can comprise power and control circuitry configured to selectively activate the repeater resonators to ensure continuous power to the wirelessly powered capsule endoscope as it moves through a gastrointestinal tract of a person. The power supply resonator can comprise coils that wrap around a person and are designed to accommodate a waist size of healthy, overweight, and obese people. The capsule enclosure can comprise a non-lossy material, the device resonator can comprise at least one device resonator coil, and the wirelessly powered capsule endoscope can comprise a shield between the at least one device resonator coil and at least a portion of the electronics. Moreover, the at least one device resonator coil can be wound helically around an axis of the wirelessly powered capsule endoscope.

According to another aspect, a wirelessly powered capsule endoscope comprises: a capsule enclosure comprising a non-lossy material; a device resonator, comprising at least one device resonator coil, configured to capture an oscillating magnetic field; and at least one electronic component, comprising power and control circuitry, held within the capsule enclosure and configured to obtain power via the captured oscillating magnetic field.

The at least one device resonator coil can be wound helically around an axis of the capsule endoscope. The endoscope can comprise a shield between the at least one device resonator coil and the at least one electronic component. The shield can be made of magnetic material, copper, and/or aluminum.

The at least one device resonator coil can be wound in a plane parallel to a minor axis of the capsule endoscope. The power and control circuitry can be configured to tune the device resonator. In addition, the endoscope can comprise a matching network.

According to another aspect, a power source for a wirelessly powered capsule endoscope system to be used with a person, the power source comprises: at least one source resonator; at least one repeater resonator; and a power and control circuitry; wherein the at least one source resonator and the at least one repeater resonator are positioned so as to be placed along an extent of a gastrointestinal tract of the person; and wherein the power and control circuitry is configured to activate and deactivate the at least one source resonator and the at least one repeater resonator.

The at least one source resonator coil can be part of clothing or a piece of furniture. The at least one repeater resonator can comprise two or more repeater resonators, and the power and control circuitry can be configured to selectively activate the two or more repeater resonators to ensure continuous power to a wirelessly powered capsule endoscope as it moves through the gastrointestinal tract of the person. In addition, the at least one source resonator can be configured to transfer 10 mW or 20 mW of power.

### DESCRIPTION OF DRAWINGS

FIG. 1 shows an embodiment of a capsule endoscope.

FIGS. 2A and 2B show embodiments of an external receiver for a capsule endoscope.

FIG. 3 shows an embodiment of a wirelessly powered capsule endoscope system.

FIG. 4 shows an embodiment of a model of a wirelessly powered capsule endoscope.

FIGS. 5A-5B show embodiments of a wirelessly powered capsule endoscope.

FIGS. 6A-6D show embodiments of a wirelessly powered capsule endoscope.

FIG. 7 shows an embodiment of a model of a source resonator for a wirelessly powered capsule endoscope.

FIG. 8 shows representations of human waist sizes.

FIGS. 9A-9C show embodiments of a source for a wirelessly powered capsule endoscope.

FIGS. 10A and 10B show embodiments of a source for a wirelessly powered capsule endoscope.

FIGS. 11A and 11B show embodiments of a source for a wirelessly powered capsule endoscope.

### DETAILED DESCRIPTION

Wireless energy transfer systems described herein may be implemented using a wide variety of resonators and resonant objects. As those skilled in the art will recognize, important considerations for resonator-based power transfer include resonator efficiency and resonator coupling. Extensive discussion of such issues, e.g., coupled mode theory (CMT), coupling coefficients and factors, quality factors (also referred to as Q-factors), and impedance matching is provided, for example, in U.S. patent application Ser. No. 13/278,993 published on Sep. 20, 2012 as US 2012/0235501 and entitled “Multi-resonator wireless energy transfer for medical applications,” and U.S. patent application Ser. No. 12/722,050 published on Jul. 22, 2010 as US 2010/0181843 and entitled “Wireless energy transfer for refrigerator application,” both of which are incorporated by reference in their entirety as if fully set forth herein.

### Introduction

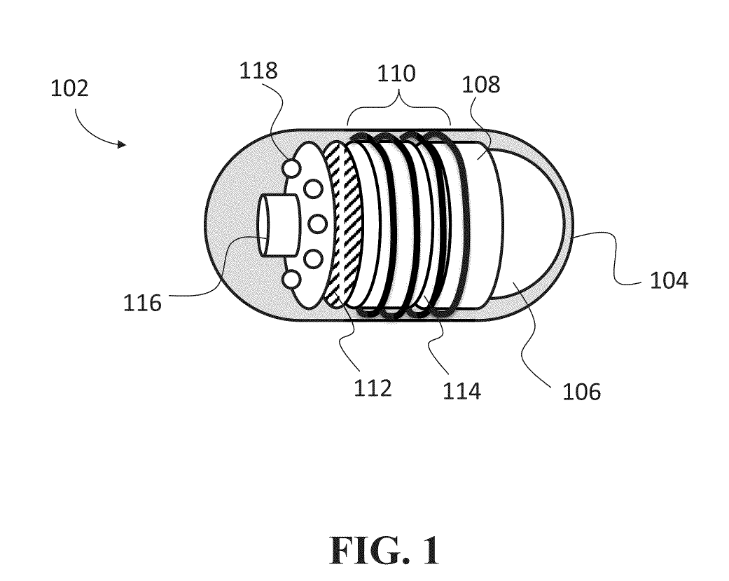

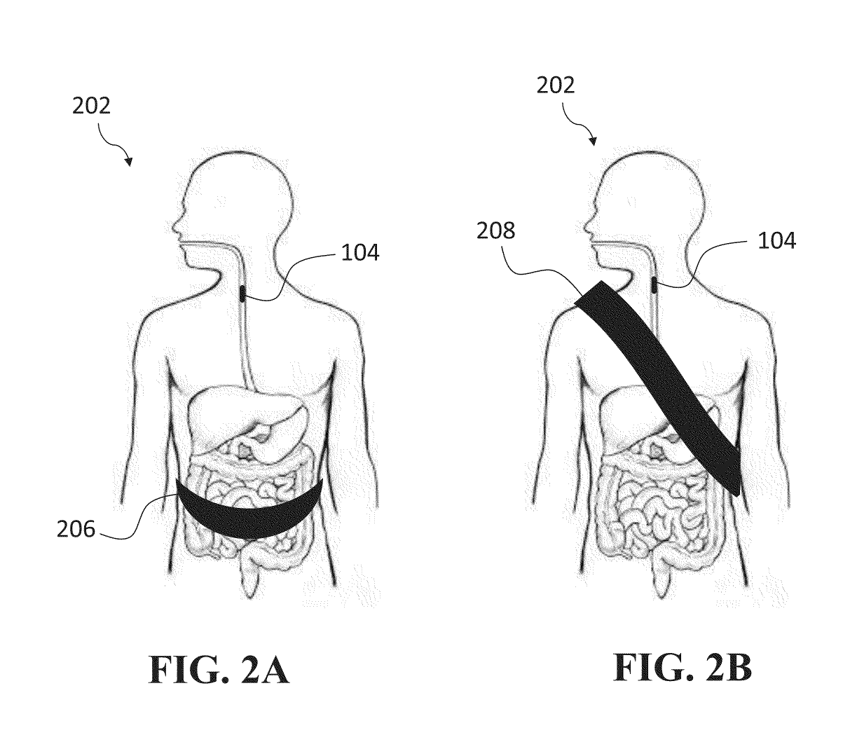

Capsule endoscopy utilizes a swallowed capsule to examine the interior of the gastrointestinal tract. Such devices may comprise at least one battery and at least one camera, and a means to store the recorded images for later retrieval or a means to transmit the recorded images to a receiver, most likely located outside the body. The endoscopic capsules may also comprise additional cameras, energy sources, sensors, lights, and the like. FIG. 1 shows an exemplary embodiment of a wirelessly powered capsule endoscope. The endoscope 102 comprises a camera 116, LEDs 118, control/processor board 112, one or more batteries 108, an antenna 106, device electronics 114 and a device resonator 110 contained in enclosure 104. The enclosure 104 may be made of plastic, polymer, or other non-lossy material that is safe to ingest and seals the components of the endoscope 102 from the outside environment. Lossy materials include metals and other materials which may increase losses in the oscillating magnetic field. The camera 116 may be used to photograph or video a patient's gastrointestinal tract and transmit the images via the antenna 106 to a receiver. FIGS. 2A-2B show exemplary embodiments of a receiver belt 206 worn around the waist, and a receiver harness 208 worn over the shoulder of a person 202. The receiver is used to receive information such as images and video from the capsule endoscope. The belt 206 or shoulder harness 208 may also house a wireless power source to supply power to the wirelessly powered capsule endoscope.

Development of a wirelessly powered capsule endoscope may allow for more power to be delivered to the module, and reduce or eliminate the need for integrated batteries, allowing more space for other capsule components. Eliminating or reducing the size of the one or more on-board energy storage devices may allow for more sensors, improved cameras, and more functionality in the capsule. For example, the quality of the recorded images and data and/or the amount of recorded images and or data may be limited by the amount of power available from the batteries and/or energy storage units in the capsule.

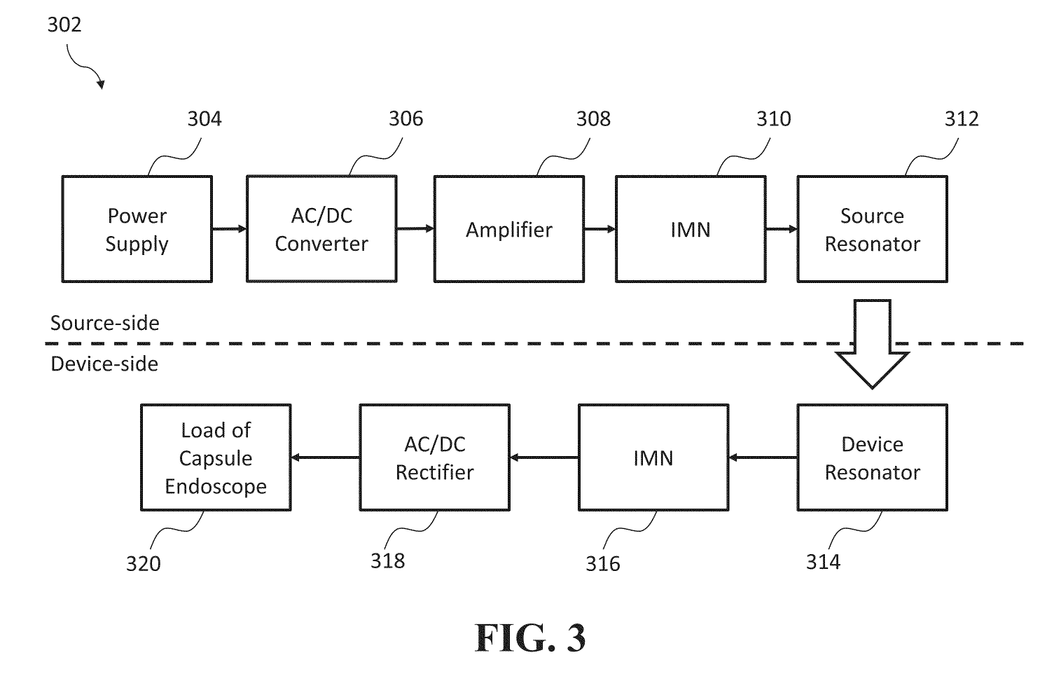

FIG. 3 shows an exemplary embodiment of a wirelessly powered capsule endoscope system 302. The system 302 may comprise a source to wirelessly transmit power to the device or capsule endoscope. The source may comprise a power supply 304, such as alternating current (AC) mains, battery, solar panel, and the like, as well as electronics 306 to convert AC to direct current (DC), an amplifier 308, an impedance matching network (IMN) 310, and one or more source resonators 312. The device may comprise one or more device resonators 314, an impedance matching network (IMN) 316, a rectifier 318, and a load 320. The load may be a battery, processor, camera, lights, another energy sink of the capsule endoscope, or a combination of these.

In some embodiments, the wirelessly powered capsule endoscope system may be optimized to have an operating frequency of approximate 250 kHz or more. In some embodiments, the operating frequency may be 6.78 MHz, 13.56 MHz or more. In some embodiments, the wirelessly powered capsule endoscope may have power consumption levels of less than 10 mW, 20 mW, or more.

### Device

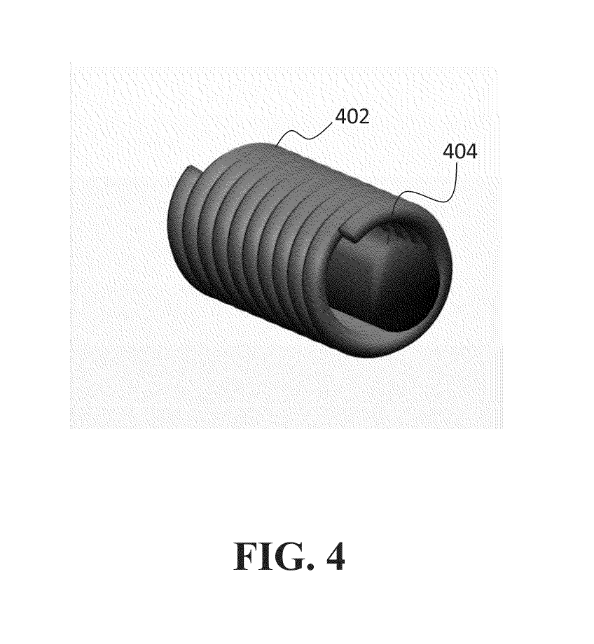

In exemplary embodiments, a wirelessly powered capsule endoscope may comprise a device resonator and device electronics. FIG. 4 shows an exemplary embodiment of a model of a wirelessly powered capsule endoscope. The wirelessly powered capsule endoscope may comprise a device resonator 402 that may be wound around a piece of magnetic material 404. The resonator 402 and magnetic material 404 may be integrated into a capsule resonator, such as into the enclosure or into the interior of a capsule resonator. The magnetic material may be used to reduce losses in magnetic field by shielding lossy elements such as metal. In exemplary embodiments, the associated device electronics may be inside of the capsule endoscope. The device electronics may comprise a rectifier 318 and matching network 316. The device electronics may comprise power and control circuitry that controls and tunes the operation of the device resonator and/or the matching network. In embodiments, for a capsule of approximate dimensions 26 mm by 11 mm, a helical resonator coil may be designed to have a diameter of about 11 mm by a length of about 20 mm and magnetic material of length 22 mm.

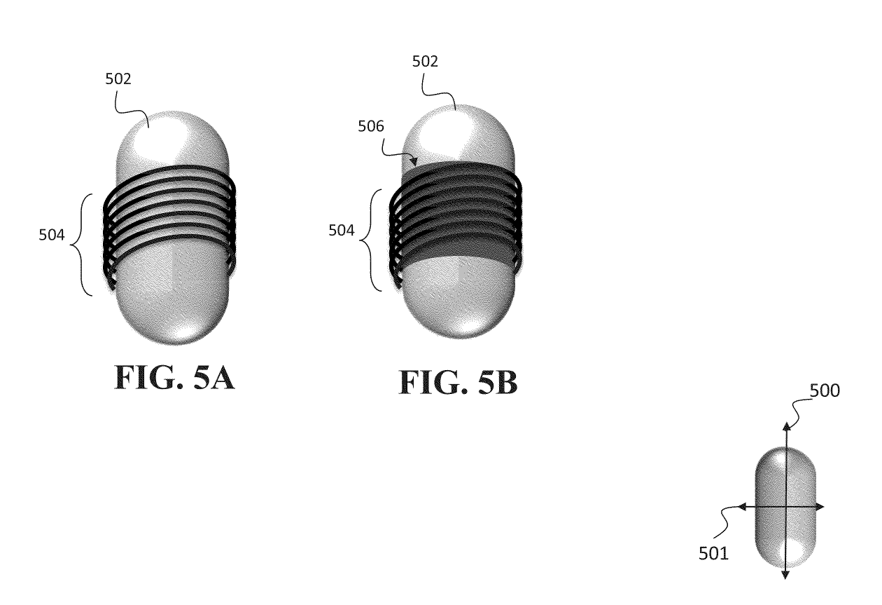

FIGS. 5A-5B show embodiments of device resonators and shields. In embodiments, a device resonator may be integrated into the outer enclosure of the capsule endoscope. In embodiments, a device resonator may reside inside the outer enclosure of the capsule endoscope. FIG. 5A shows an exemplary embodiment of a device resonator 504 wound helically around the major axis 500 of the capsule 502. FIG. 5B shows an exemplary embodiment of a device resonator 504 wound helically over a shield 506. The shield may be integrated into the enclosure of the capsule endoscope. In embodiments, it may be beneficial to cover a part of the capsule with a shield. This may be useful to allow for wireless communication signals from the antenna inside the capsule to reach the receiver outside of the capsule. In other embodiments, it may be beneficial to cover all or most of a capsule with magnetic material to prevent losses in the metallic or lossy elements of a capsule endoscope (e.g. metallic parts such as the electronics internal to the capsule endoscope). In embodiments, the shield may be made of magnetic material, copper, aluminum, and the like. In embodiments, the device resonator 504 is integrated into the enclosure of the capsule endoscope. In embodiments, the device resonator 504 is wound helically around the minor axis 501 of the capsule 502.

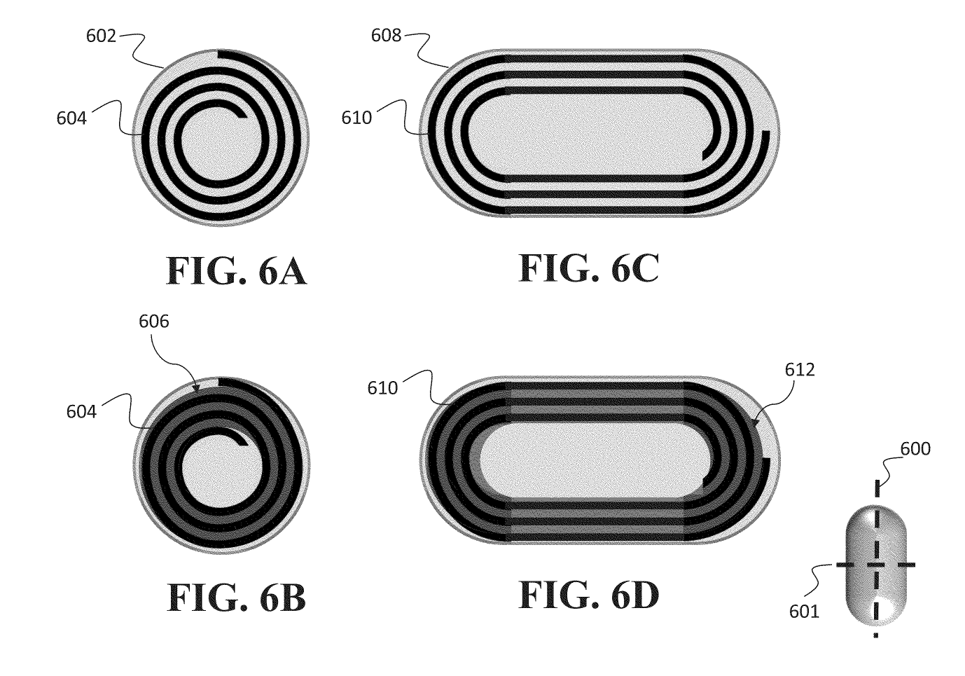

FIGS. 6A-6D show embodiments of device resonators and shields. FIG. 6A shows an exemplary embodiment of a device resonator 604 wound in a plane 602 of a cross-section 601 of a capsule endoscope. FIG. 6B shows an exemplary embodiment of a device resonator 604 wound over a shield 606 in a cross-section 601 of a capsule endoscope. The shield 606 may cover all or part of the plane 602 of a cross-section 601. In embodiments, the shield may be made of magnetic material, copper, aluminum, and the like. The shield may be used to prevent losses in the lossy or metallic elements of a capsule endoscope. FIG. 6C shows an exemplary embodiment of a device resonator 610 wound in a plane 608 of a cross-section 600 of a capsule endoscope. FIG. 6D shows an exemplary embodiment of a device resonator 610 wound over a shield 612 in a cross-section of a capsule endoscope. The shield 612 may cover all or part of the plane 608 of a cross-section 600. In some embodiments, the device resonator may not fully reside in a plane of a cross-section of a capsule endoscope and may partially or fully follow the curvature of the capsule endoscope shape. The resonators in FIGS. 6A and 6C may be combined in a single capsule endoscope. Two device resonators 604 and 610 may allow for freedom of orientation with respect to the source resonator. The capsule endoscope may undergo various orientations as it travels through the gastrointestinal tract and may best couple with a source resonator in one axis over another at any given time. This may increase the efficiency of power transfer.

### Source

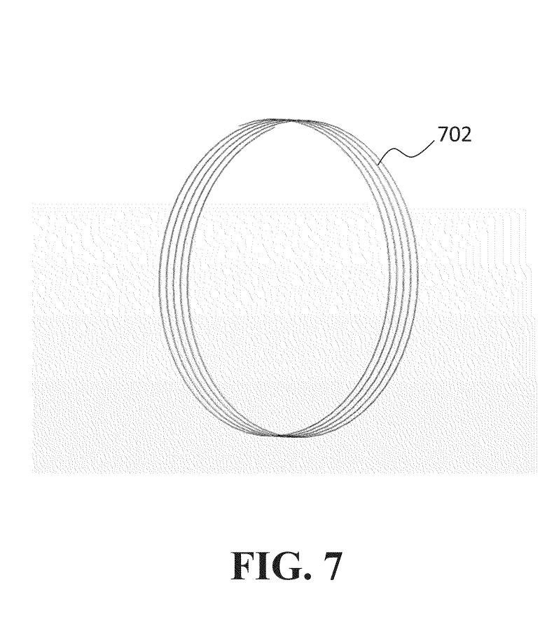

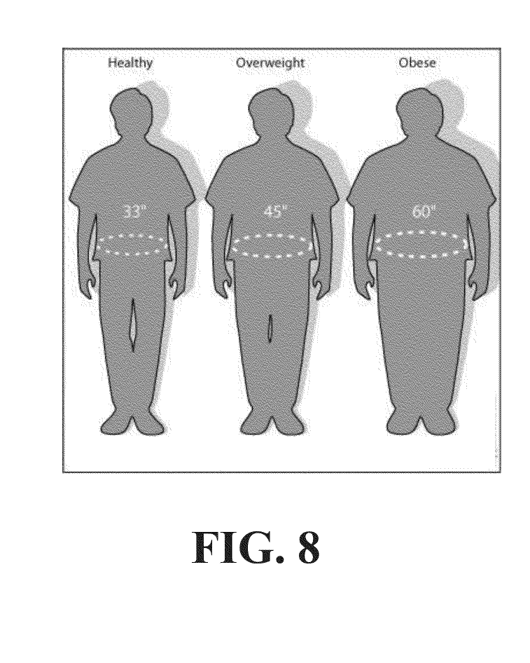

In exemplary embodiments, a source may comprise source electronics and at least one source resonator. Source electronics may comprise power and control circuitry to control and tune the matching network and/or source resonator. In embodiments, a patient (or person or animal swallowing the capsule) may wear one or more source resonators around their abdomen. FIG. 7 shows an exemplary embodiment of a source resonator coil 702. The size of the source resonator coil may be determined by the circumference of the patient or subject of the endoscopy. For example, FIG. 8 shows a representation of a range of circumferences a source may have to accommodate (“Healthy” person with a waist of about 33 inches, “Overweight” person with a waist of about 45 inches, and “Obese” person with a waist of about 60 inches). In embodiments, there may be different sized sources chosen according to the patient size. In embodiments, the different sized source resonators may be switched or toggled depending on the size of the patient. The source resonator coil may be encased in a solid structure or embedded in a flexible material such as a vest or belt worn by the patient or a flexible resonator taped to the body.

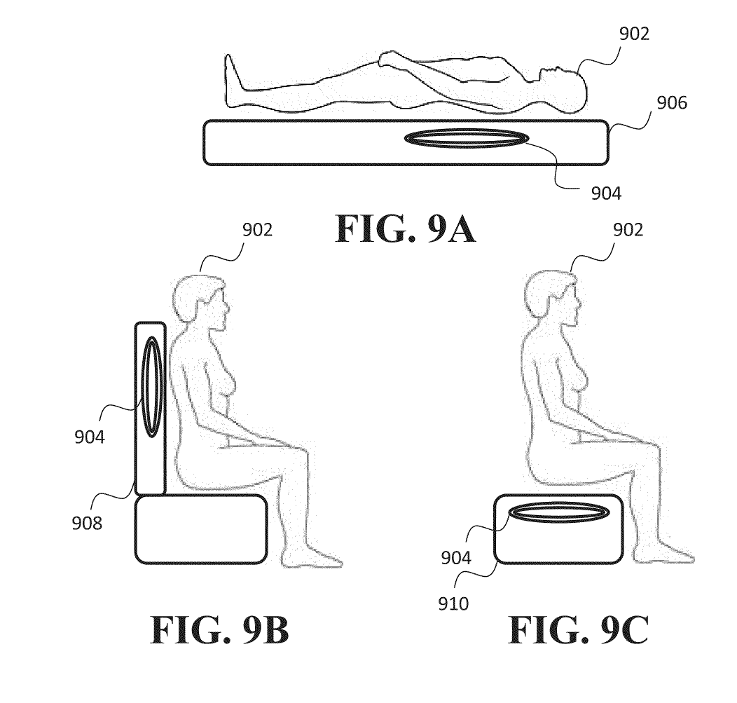

In exemplary embodiments, a source may be embedded in furniture, bedding, chairs, beds, couches, beds, and the like to allow for convenient powering or recharging of the endoscopic capsule throughout the day. FIG. 9A-9C show configurations of a source 904 integrated into furniture such as a bed 906, the back of a chair 908, and a chair seat 910. In exemplary embodiments, a repeater may also be integrated into furniture or clothing. For example, a source may be placed in a chair seat and a repeater may be placed in the back of the chair to increase the efficiency of power transfer.

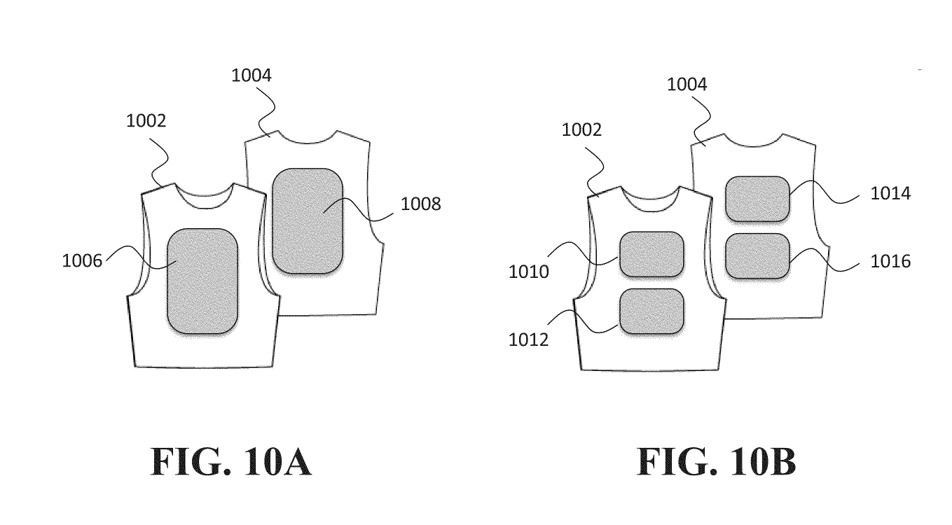

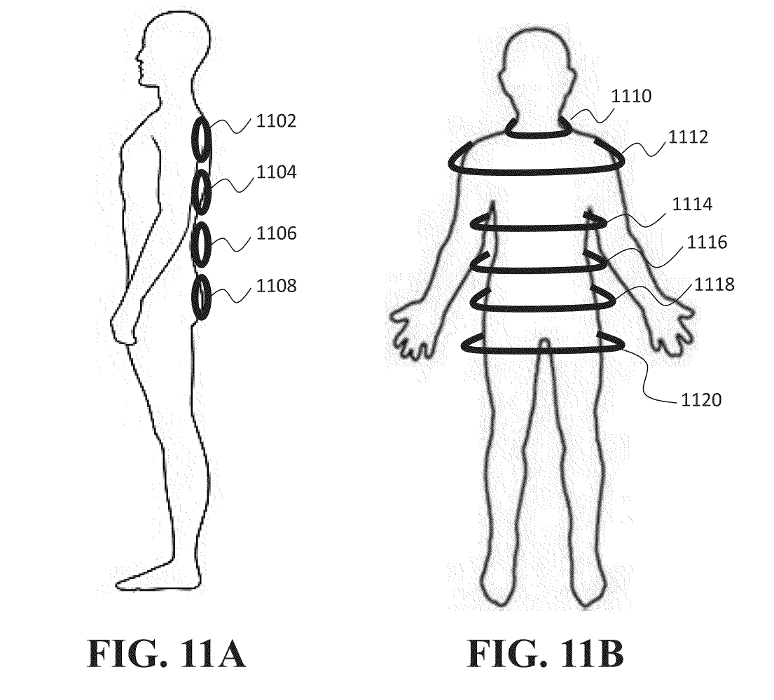

In exemplary embodiments, a source may be integrated into clothing, such as a shirt, vest, jacket, coat, bib, belt, suspenders, dress, and the like. FIG. 10A shows a representation of a vest comprising one or more source resonators 1006, 1008. In embodiments, clothing may comprise one or more source resonators in the front 1002 and/or back 1004 of the clothing item. FIG. 10B shows a representation of a vest comprising more than one source resonator 1010, 1012, 1014, 1016 in the front 1002 and/or back 1004. In exemplary embodiments, a source resonator may be coupled to multiple repeaters positioned in different locations around the body or integrated into clothing. For example, a number of repeater coils may be placed along the body to ensure continuous power to the capsule as it moves through the digestive tract. The vest shown in FIG. 10B may comprise a source resonator 1010 and several repeater resonators 1012, 1014, 1016. The vest may also comprise more than one source resonator 1010 and 1014 and repeater resonators 1012 and 1016. FIGS. 11A-11B show one or more source resonators and repeater resonators 1102-1120 placed along a patient's abdomen, parallel to the gastrointestinal tract. FIG. 11A shows resonators 1102, 1104, 1106, 1108 along the back of a patient; these resonators may be either source or repeater and may be activated as the wirelessly powered capsule endoscope moves throughout the patient's body. For example, the power and control circuitry as part of the source may activate and/or deactivate source resonators and repeaters as capsule moves through the patient's body. FIG. 11B shows resonators 1110, 1112, 1114, 1116, 1118, 1120 wrapped around a patient, such as in an item of clothing. The embodiment shown in FIG. 11A may be housed in clothing or be embedded in a bed, such as a hospital bed.

### Other Embodiments

In exemplary embodiments, there may be multiple capsules used in the endoscopy. In such a case, each of these capsules may each comprise at least one device resonator coil. One or more source resonators may be used to provide wireless power to these capsule endoscopes.

In exemplary embodiments, a wireless energy transfer system may be used to power other ingestible or swallowable pills, capsules, imaging tools, sensors and the like. A pill may comprise one or more resonators and electronics. The pill may then receive power via a source that is outside of the patient's body. Multiple pills or capsules may be supported with one or more source resonators or repeater resonators. In embodiments, the pills may send communication such as the temperature of the patient's body, report the health of an athlete or soldier, take images or video of the inside of the subject's body, measure and report the compliance of a patient taking pills, deploy medicine, and the like.

In exemplary embodiments, a means of communication may be integrated into the capsule endoscope. This may include in-band communication or out-of-band communication. In-band communication may be a means of exchanging information over the wireless power transfer signal. Out-of-band communication may include Bluetooth, Wi-Fi, radio, and the like. Extensive discussion of communication in a wireless power transfer system is provided, for example, in U.S. patent application Ser. No. 13/222,915 published on Mar. 15, 2012 as US Patent Publication US 2012/0062345 A1 and entitled “Low resistance electrical conductor”.

In exemplary embodiments, wirelessly transmitted power may be used to power a drive system of the capsule endoscope. A drive system may comprise a motor which may be used to speed up or slow down the capsule through the gastrointestinal tract. In embodiments, the power transmitted may be increased to speed up the capsule and decreased to slow down the capsule.

A number of embodiments have been described. Nevertheless, it will be understood that various modifications may be made without departing from the spirit and scope of the disclosure. Accordingly, other embodiments are within the scope of the following claims.

What is claimed is:

1. A wirelessly powered capsule endoscope comprising:
a capsule enclosure comprising a non-lossy material;
a device resonator, comprising at least one device resonator coil, configured to capture an oscillating magnetic field; and
at least one electronic component, comprising power and control circuitry, held within the capsule enclosure and configured to obtain power via the captured oscillating magnetic field.

2. The endoscope of claim 1, wherein the at least one device resonator coil is wound helically around an axis of the capsule endoscope.

3. The endoscope of claim 1, comprising a shield between the at least one device resonator coil and the at least one electronic component.

4. The endoscope of claim 3, wherein the shield is made of magnetic material.

5. The endoscope of claim 3, wherein the shield is made of copper.

6. The endoscope of claim 3, wherein the shield is made of aluminum.

7. The endoscope of claim 1, wherein the at least one device resonator coil is wound in a plane parallel to a minor axis of the capsule endoscope.

8. The endoscope of claim 1, wherein the power and control circuitry is configured to tune the device resonator.

9. The endoscope of claim 1, comprising a matching network.

10. A power source for a wirelessly powered capsule endoscope system to be used with a person, the power source comprising:
at least one source resonator;
at least one repeater resonator; and
a power and control circuitry;
wherein the at least one source resonator and the at least one repeater resonator are positioned so as to be placed along an extent of a gastrointestinal tract of the person; and
wherein the power and control circuitry is configured to activate and deactivate the at least one source resonator and the at least one repeater resonator.

11. The power source of claim 10, wherein the at least one source resonator coil is part of clothing.

12. The power source of claim 10, wherein the at least one source resonator is part of a piece of furniture.

13. The power source of claim 10, wherein the at least one repeater resonator comprises two or more repeater resonators, and the power and control circuitry is configured to selectively activate the two or more repeater resonators to ensure continuous power to a wirelessly powered capsule endoscope as it moves through the gastrointestinal tract of the person.

14. The power source of claim 10, wherein the at least one source resonator is configured to transfer 10 mW of power.

15. The power source of claim 10, wherein the at least one source resonator is configured to transfer 20 mW of power.

16. A wirelessly powered capsule endoscope system comprising:
a wirelessly powered capsule endoscope comprising a camera, a light source, electronics, an antenna, a device resonator, and a capsule enclosure; and
a power supply resonator;
wherein the power supply resonator is configured and arranged to resonantly couple with the device resonator to provide power to the wirelessly powered capsule endoscope via an oscillating magnetic field.

17. The wirelessly powered capsule endoscope system of claim 16, wherein the power supply resonator comprises a source resonator and repeater resonators, and the system comprises power and control circuitry configured to selectively activate the repeater resonators to ensure continuous power to the wirelessly powered capsule endoscope as it moves through a gastrointestinal tract of a person.

18. The wirelessly powered capsule endoscope system of claim 16, wherein the power supply resonator comprises coils that wrap around a person and are designed to accommodate a waist size of healthy, overweight, and obese people.

19. The wirelessly powered capsule endoscope system of claim 16, wherein the capsule enclosure comprises a non-lossy material, the device resonator comprises at least one device resonator coil, and the wirelessly powered capsule endoscope comprises a shield between the at least one device resonator coil and at least a portion of the electronics.

20. The wirelessly powered capsule endoscope system of claim 19, wherein the at least one device resonator coil is wound helically around an axis of the wirelessly powered capsule endoscope.
